(19) United States
(12) Patent Application Publication
(10) Pub. No.: US 2012/0053669 A1
(43) Pub. Date: Mar. 1, 2012

(54) Stent Delivery Device and Methods of Use Thereof

(75) Inventors: Dennis Creedon, Sandwich, MA (US); Lewis H. Marten, Westwood, MA (US)

(73) Assignee: E. Benson Hood Laboratories, Pembroke, MA (US)

(21) Appl. No.: 13/222,822

(22) Filed: Aug. 31, 2011

Related U.S. Application Data
(60) Provisional application No. 61/378,813, filed on Aug. 31, 2010.

(51) Int. Cl. A61F 2/82; B23P 19/00

(52) U.S. Cl. 623/1.11; 29/700

(57) ABSTRACT

Device for delivering an airway stent to patients and methods of using the stent. The device may comprise a sheath to position the stent in the airway, and a fiber scope or ventilator to assist in delivering the stent. The device may be used to deliver, for example, a tracheal or nasal stent. Further, a device for folding stents and a device for loading folded stents into a stent delivery device.

### CROSS-REFERENCES TO RELATED APPLICATIONS

This application claims priority to U.S. provisional application Ser. No. 61/378,813, filed on Aug. 31, 2010, the entirety of which is incorporated herein by reference.

### FIELD OF THE INVENTION

The present invention relates to a device used to deliver airway stents to patients. The device may comprise a sheath to position the stent in the airway and a fiber scope, and may allow for ventilation during the process of delivering the stent. The device may be used to deliver, for example, a tracheal or sinus stent, and the subsequent repositioning of the stent.

### BACKGROUND OF THE INVENTION

Airway management devices may be used for a variety of reasons including facilitation of speaking and breathing following a laryngectomy, promotion of healing in the patient, provision of an access point for forced ventilation of a patient, and assistance in supplying oxygen to augment normal breathing. In particular, airway stents such as tracheal and sinus stents are often used to relieve areas of obstruction or stenosis in the airways, prevent encroachment of masses such as a tumour into the airway, or support an airway during resection. Sinus stents are also used to facilitate drainage postoperatively.

In order for airway stents to provide relief to areas of stenosis and to maintain the airway, it is important that the stent is accurately positioned. Physicians who perform such procedures often use angled or straight non-serrated giraffe forceps, and are without the aid of direct vision. This can result in improper placement of the stent and repeated intubations, and can lead to a failure to remedy the stenosis or to prevent further obstruction of the airway; consequently, the stent may have to be removed and a new stent may have to be administered.

A means to more accurately place a stent within the airway and reposition the stent during the initial administration would improve the success rate of airway stents, save costs of repeating procedures, and avoid additional trauma to patients.

### SUMMARY OF THE INVENTION

The present invention provides devices used to deliver airway stents to patients, and methods of using the devices.

One aspect of the present invention relates to devices for delivering a stent to a site in the airway of a patient in need thereof. In certain embodiments, the device may comprise (a) a sheath comprising: a proximal region; a distal region; a middle region therebetween; a lumen extending through the sheath, such that the lumen comprises a diameter; a superior aspect comprising a channel extending the length of the sheath, such that the channel comprises an inner diameter; and an inferior aspect. In certain embodiments, the device may further comprise an instrument comprising an outer diameter, wherein the outer diameter of the instrument is less than the diameter of the channel to allow the instrument to fit inside the channel.

In some embodiments, the instrument may be a fiber scope or a ventilator. In certain embodiments, the fiber scope may be a bronchoscope. In some embodiments, the fiber scope may be rigid or flexible.

In certain embodiments, the diameter of the lumen of the sheath can fit the outer diameter of a tracheal stent.

In certain embodiments, the distal region may comprise a beveled distal end. In some embodiments, the sheath comprises stainless steel.

In certain embodiments, the device may further comprise a stent delivery pusher comprising: a proximal region, a distal region, and a middle region therebetween, and an outer diameter, such that the outer diameter of the stent delivery pusher may be less than the diameter of the lumen of the sheath to allow the stent delivery pusher to fit inside the lumen of the sheath.

In some embodiments, the stent delivery pusher may be a rod or tube. In certain embodiments, the stent delivery pusher may comprise removable tips. In some embodiments, these tips may be attached to the stent delivery pusher by screw, snap-fit, or adhesive. In certain embodiments, the tips may comprise an outer diameter greater than the outer diameter of the stent delivery pusher, but the outer diameter is less than the diameter of the lumen of the sheath.

In some embodiments, the stent delivery pusher may comprise graduated markings.

In some embodiments of the invention, the diameter of the lumen of the sheath can fit the outer diameter of a sinus stent. In certain embodiments, the sheath may comprise a curvature. In some embodiments, the curvature may be in the distal region. In certain embodiments, the channel may be within the superior aspect of the sheath. In some embodiments, the channel may be outside of the superior aspect of the sheath.

Another aspect of the invention relates to methods of delivering a stent to a site in an airway of a patient in need thereof. In certain embodiments, the method may comprise (1) placing a stent delivery device of the invention in the airway of the patient, in which a stent is in the lumen of the sheath of the device; (2) positioning the distal end of the instrument of the device inside the airway by viewing the inside of the airway with the instrument, and moving the instrument in the airway until the distal end of the instrument is adjacent to the site in the airway at which the stent is to be placed; (3) depositing the stent at the site in the airway; and (4) removing the instrument from the airway.

In some embodiments, the stent is deposited by a stent delivery pusher. For example, the stent delivery pusher may push the stent out of the sheath of the stent delivery device.

In certain embodiments, graduated markings on the stent delivery pusher may guide how far to insert the stent delivery pusher into the sheath of the stent delivery device. In some embodiments, the site in the airway may be a stenosis.

A further aspect of the invention relates to stents for placement into a stent delivery device. In certain embodiments, the stent may comprise a sheath comprising: a proximal region, a distal region, and a middle region therebetween, a lumen extending through the sheath, such that the lumen comprises a diameter. The outer surface of the stent may comprise a groove.

Another aspect of the invention relates to stent folding devices for folding a stent for placement into a stent delivery device. In certain embodiments, the stent folding device comprises a sheath comprising: a proximal region, a distal region, a middle region therebetween, and a lumen extending through the sheath. In some embodiments, the lumen comprises a diameter. In certain embodiments, the diameter of the lumen in the proximal region of the sheath is greater that the diameter of the lumen in the middle region of the sheath. In some embodiments, the lumen in the distal region of the sheath is configured to receive an end of a stent delivery device.

In certain embodiments, the stent folding device further comprises one or more fins inside the lumen.

In some embodiments, the stent folding device may further comprise a cylindrical stent folding pusher comprising an outer diameter that is less than the diameter of the lumen, and outer surface, and a groove on the outer surface that is configured to fit the one or more fins in the lumen of the sheath.

In some embodiments, the groove on the outer surface of the stent of the present invention is configured to fit the one or more fins in the lumen of the sheath.

An additional aspect of the present invention is methods of inserting a stent into stent delivery device. In some embodiments, the method involves folding the stent. In some embodiments, the method involves the use of the stent folding device of the present invention. In certain embodiments, the method involves the use of the stent delivery device of the present invention. In particular embodiments, the method involves both the use of the stent folding device of the present invention and the stent delivery device of the present invention.

In certain embodiments, the method of folding a stent for insertion into a stent delivery device comprises (1) inserting an end of the stent delivery device into the lumen of the sheath of the stent folding device, wherein the end of the stent delivery device is inserted into the distal region of the sheath; (2) inserting the stent into the lumen of the sheath, wherein the stent is inserted into the proximal region of the sheath; (3) moving the stent through the lumen from the proximal region of the sheath to the distal region of the sheath and through the end of the stent delivery device; and (4) removing the end of the stent delivery device from the lumen of the sheath of the stent folding device.

In some embodiments, the stent is moved through the lumen of the sheath of the stent folding device using a stent folding pusher. In certain embodiments, the stent folding pusher is inserted into the sheath of the stent folding device, and pressure is applied to push the stent through the lumen.

Yet another aspect of the invention relates to stent loading devices for loading a folded stent into a stent delivery device. In certain embodiments, the stent loading device may comprise a sheath, which comprises a proximal region, a distal region, a middle region therebetween, and a lumen extending through the sheath. In some embodiments, the lumen comprises a diameter. In certain embodiments, the diameter of the lumen in the proximal region of the sheath is greater that the diameter of the lumen in the middle region of the sheath.

In some embodiments, the proximal region of the sheath is configured to be inserted into a stent folding device. In certain embodiments, the lumen in the distal region of the sheath is configured to receive an end of a stent delivery device.

In some embodiments, the stent loading device may further comprise a cylindrical stent loading pusher comprising an outer diameter that is less than the diameter of the lumen.

An aspect of the present invention relates to methods of inserting a folded stent into stent delivery device. In embodiments of the invention, the method involves using a device to transport the stent from a stent folding device to the stent delivery device. In certain embodiments, the method involves the use of the stent loading device of the present invention. In certain embodiments, the method involves the use of the stent delivery device of the present invention. In some embodiments, the method involves the use of a stent loading device. In certain embodiments, the method involves the use of the stent loading device of the present invention. In particular embodiments, the method involves the use of the stent loading device of the present invention, the stent folding device of the present invention and the stent delivery device of the present invention.

In certain embodiments, the method may comprise (1) inserting the proximal region of the sheath of a stent loading device into a stent folding device, wherein the stent folding device contains a folded stent; (2) moving the folded stent from the stent folding device into the proximal region of the sheath; (3) inserting an end of the stent delivery device into the lumen of the sheath, wherein the end of the stent delivery device is inserted into the distal region of the sheath; (4) pushing the stent through the lumen of the sheath from the proximal region to the distal region of the sheath and through the end of the stent delivery device; (5) removing the end of the stent delivery device from the lumen of the sheath of the stent loading device.

In some embodiments, the stent is moved through the lumen of the sheath of the stent folding device using a stent folding pusher. In certain embodiments, the stent folding pusher is inserted into the sheath of the stent folding device, and pressure is applied to push the stent through the lumen.

In some embodiments, the stent is moved through the lumen of the sheath of the stent loading device using a stent loading pusher. In certain embodiments, the stent loading pusher is inserted into the sheath of the stent folding device, and pressure is applied to push the stent through the lumen.

### BRIEF DESCRIPTION OF THE DRAWINGS

For a more complete understanding of the invention, reference is made to the following description and accompanying drawings, as follows:

FIGS. 1a-c show a tracheal stent delivery device according to certain embodiments of the present invention. FIG. 1a is a side view of the stent delivery device, FIG. 1b is a proximal end view of the stent delivery device, and FIG. 1c is a cut-away end view at section A-A of the stent delivery device in FIG. 1a.

FIGS. 2a-b show a tracheal stent delivery device according to certain embodiments of the present invention. FIG. 2a is a magnified side view of the distal region of the sheath of the stent delivery device, and FIG. 2b is a cut-away end view at section A-A of the sheath of the stent delivery device in FIG. 2a.

FIGS. 3a-c show a tracheal stent delivery device according to certain embodiments of the present invention, wherein the sheath of the stent delivery device comprises fenestrations. FIG. 3a is a side view of the device, FIG. 3b is a proximal end view of the device, and FIG. 3c is a distal end view of the device.

FIGS. 4a-c show a sinus stent delivery device according to certain embodiments of the present invention, wherein the device comprises a curvature. FIG. 4a is a side view of the device, FIG. 4b is a proximal end view of the device, and FIG. 4c is a cut-away end view at section A-A of the device in FIG. 4a.

FIGS. 5a-b show a sinus stent delivery device according to certain embodiments of the present invention, wherein the device comprises a curvature and a fiber scope. FIG. 5a is a side view of the device, and FIG. 5b is a proximal end view of the device.

FIGS. 6a-c show a sinus stent delivery device according to certain embodiments of the present invention, wherein the device comprises a curvature and a channel as a separate tube. FIG. 6a is a side view of the device, FIG. 6b is a proximal end view of the device, and FIG. 6c is a cut-away end view of section A-A of the device in FIG. 6a.

FIGS. 7a-c show a sinus stent delivery device according to certain embodiments of the present invention, wherein the device comprises a curvature and a channel as a separate tube having one or more fenestrations on the distal end. FIG. 7a is a side view of the device, FIG. 7b is a proximal end view of the device, and FIG. 7c is a magnified view of the distal end of the device.

FIGS. 8a-b show a rubber cap according to certain embodiments of the present invention. FIG. 8a is a side view of the cap, and FIG. 8b is a proximal end view of the cap.

FIGS. 9a-b show a rubber cap for insertion into the proximal end of a tracheal stent delivery device according to embodiments of the present invention. FIG. 9a is a side view of the cap and device, and FIG. 9b is a proximal end view of the cap and device.

FIGS. 10a-b show a tracheal stent delivery device according to certain embodiments of the present invention, wherein the device contains a stent. FIG. 10a is a side view of the device and stent, and FIG. 10b is a proximal end view of the device and stent.

FIGS. 11a-e show a stent delivery pusher according to certain embodiments of the present invention. FIG. 11a is a side view of the stent delivery pusher. FIG. 11b is a proximal end view of the stent delivery pusher. FIG. 11c is a side view of the stent delivery pusher without a tip. FIG. 11d is a side view of a tip according to some embodiments of the invention, and FIG. 11e is a side view of a tip according to other embodiments of the invention.

FIGS. 12a-b show a stent delivery pusher that is progressing a tracheal stent through the lumen of a stent delivery device according to embodiments of the present invention. FIG. 12a is a side view, and FIG. 12b is a proximal end view.

FIGS. 13a-b show a tracheal stent delivery device and a stent delivery pusher after the stent delivery pusher has pushed a stent through the lumen of the device, according to certain embodiments of the present invention. FIG. 13a is a side view, and FIG. 13b is a proximal end view.

FIGS. 14a-e show a sheath of a stent folding device according to certain embodiments of the present invention. FIG. 14a is a cut-away side view of the sheath. FIG. 14b is a cut-away side view showing the division of the sheath into sections. FIG. 14c is a proximal end view, FIG. 14d is a distal end view, and FIG. 14e is a superior view.

FIGS. 15a-e show steps of folding a stent and inserting the folded stent into the sheath of the stent delivery device according to certain embodiments of the present invention. FIG. 15a is a cut-away side view of the sheath of the stent folding device when the stent is in the first section of the sheath. FIG. 15b is a proximal end view of the sheath of the stent folding device. FIG. 15c is a cut-away side view of the sheath of the stent folding device when the stent is bridging the second and third sections of the sheath and beginning to enter the stent delivery device. FIG. 15d is a cut-away side view of the sheath of the stent folding device when the stent is progressing further into the stent delivery device, and FIG. 15e is a distal end view of the sheath of the stent folding device and the stent.

FIGS. 16a-d show a stent folding pusher according to certain embodiments of the present invention. FIG. 16a is a cut-away side view of the stent folding pusher, FIG. 16b is a distal end view of the stent folding pusher inside the sheath of the stent folding device. FIG. 16c is a cut-away side view of the tube of the stent folder, and FIG. 16d is a cut-away side view of the stent folding pusher inside the tube of the stent folder.

FIGS. 17a-d show a sinus stent delivery device with a sinus catheter pusher according to certain embodiments of the present invention, wherein the device comprises a curvature. FIG. 17a is a cut-away side view of the device with the sinus catheter pusher inserted partially into the sinus stent delivery device, FIG. 17b is a cut-away view of section of A-A of the device of FIG. 17a, FIG. 17c is a cut-away side view of the device with the sinus catheter pusher inserted nearly fully into the sinus stent delivery device; and FIG. 17d is a proximal end view of the device of FIG. 17c.

FIGS. 18a-b show a stent delivery device with a ventilation tube according to certain embodiments of the present invention. FIG. 18a is a side view of the device, and FIG. 18b is a superior view of the device.

FIGS. 19a-c show a stent delivery device with a ventilation tube according to some embodiments of the present invention. FIG. 19a is a superior view of the device with a component of the device highlighted, FIG. 19b is a magnified superior view of the highlighted component in FIG. 19a, and FIG. 19b is a magnified side view of the highlighted component in FIG. 19a.

FIGS. 20a-b show a stent loading device according to some embodiments of the present invention. FIG. 20a is a cut-away side view of the sheath of a stent loading device, and FIG. 20b is a side view of the stent loading pusher.

FIGS. 21a-l show steps of moving a folded stent from a stent folding device to a stent delivery device using a stent loading device according to some embodiments of the invention. FIG. 21a shows a cut-away side view of the proximal end of the sheath of a stent loading device inserted into the distal end of the sheath of a stent folding device. FIG. 21b shows a cut-away side view of the devices shown in FIG. 21a, wherein a stent is positioned in the proximal region of the sheath of the stent folding device. FIG. 21c shows a proximal view of the devices shown in FIG. 21b. FIG. 21d shows a stent folding pusher inserted into the proximal end of the sheath of the stent folding device. FIG. 21e shows the stent folding pusher moving the stent from the sheath of the stent folding device into the sheath of the stent loading device. FIG. 21f shows the removal of the stent folding pusher. FIG. 21g shows the removal of the proximal end of the sheath of the stent loading device from the distal end of the sheath of the stent folding device. FIG. 21h shows a cut-away side view of the sheath of the stent loading device and a stent delivery device, in which the distal end of the sheath of the stent loading device is being inserted into the proximal end of the sheath of the stent delivery device. FIG. 21i shows a proximal view of the devices shown in FIG. 21h. FIG. 21i shows a stent loading pusher inserted into the proximal end of the sheath of the stent loading device. FIG. 21k shows insertion of a stent delivery pusher after the stent loading pusher had moved the stent from the sheath of the stent loading device into the sheath of the stent delivery device, and the stent loading pusher and the sheath of the stent loading device had been removed. FIG. 21l shows the stent delivery pusher moving the stent through the length of the sheath of the stent delivery device.

### DETAILED DESCRIPTION OF THE INVENTION

The present invention addresses current problems with deploying stents, typically silicone stents, in patients. Under current methodology, physicians and medical personnel cannot observe where the stent is being deployed, and even if scopes are used to aid physicians, these scopes will not accommodate the larger tracheal stents. To this end, the present invention advantageously permits physicians and medical personnel to directly observe how and where stents are deployed in patients.

Hence, the present invention is directed to a device that positions a stent into a patient in need thereof, confirms appropriate placement of the stent, and/or repositions the stent if not in the appropriate position. The present invention also relates to a stent folding device, a stent loading device, and an instrument for introducing a sinus catheter into the sinus cavity. Furthermore, the present invention relates to methods of using the devices described herein.

### Delivery Device

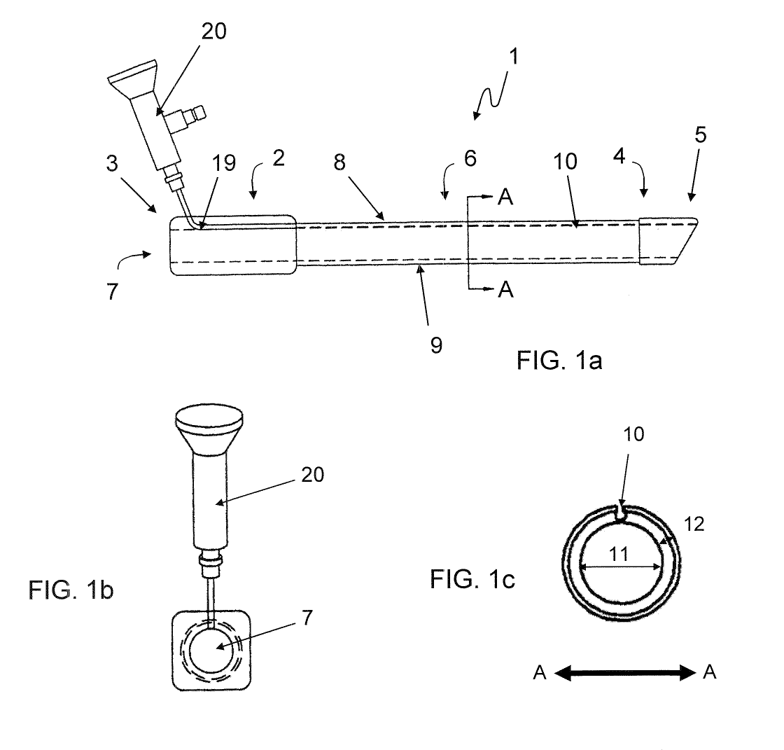

The present invention relates to a delivery device, such as a tracheal stent delivery device or a sinus stent delivery device, or a catheter delivery device. Embodiments of such delivery devices are depicted in FIGS. 1a-c, 2a-b, 3a-c, 4a-c, 5a-b, 6a-c, 7a-c, 10a-b, 13a-b, 17a-d, 18a-b, and 19a-c.

According to embodiments of the present invention, a delivery device may comprise a circular sheath 1, which comprises a proximal region 2 having a proximal end 3, a distal region 4 having a distal end 5, a middle region therebetween 6, a lumen 7 extending through the sheath 1, a superior aspect 8 of the sheath 1, and an inferior aspect 9 of the sheath 1. The superior aspect 8 may comprise a channel 10, which extends the length of the sheath 1. The lumen 7 may comprise a diameter 11. The diameter 11 may be appropriate for the diameter of stents, such as, for instance, 0.35-0.70 inches.

The length of the sheath 1 can be determined by one of ordinary skill in the art, based on anatomical measurements of the airways. For example, the sheath 1 may be about 13-16 inches in length.

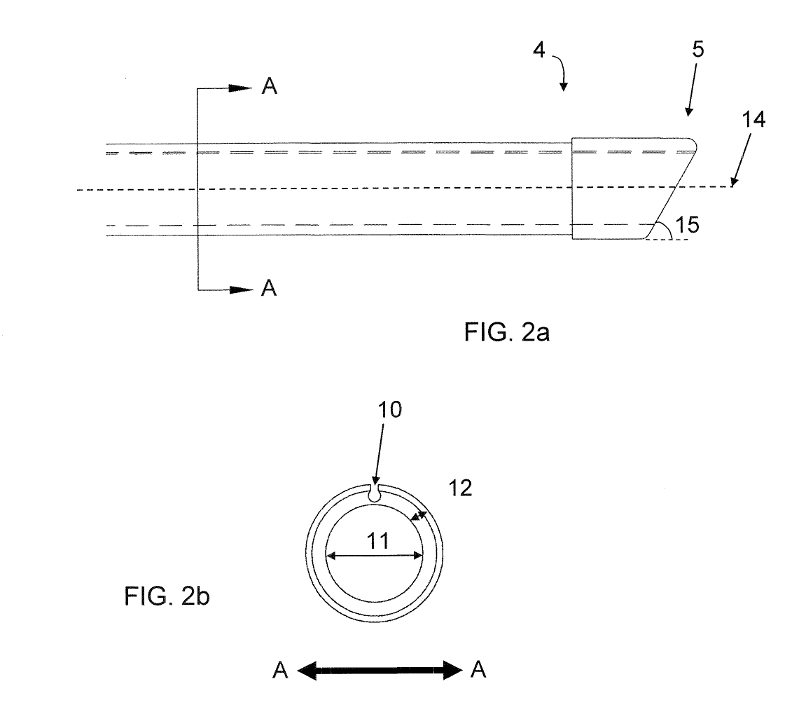

The sheath 1 may comprise thin wall extruded stainless steel or titanium. The sheath may comprise a thickness 12, i.e., distance from the outer edge of the lumen 7 to the outside of the sheath 1, as shown in FIGS. 2a-b. The thickness 12 may be uniform throughout the sheath 1 or may differ in particular regions. For example, the thickness 12 may be greater in the proximal region 2 and/or distal 4 region than in the middle region 6, as demonstrated in FIGS. 1a-c, 2a-c, and 3a-c. A proximal region 2 that is greater in thickness may be easier to clean and supports entry of flexible telescope into the channel 10. A distal region 4 that is greater in thickness forms an edge that is less sharp.

In certain embodiments, in one or more regions of the sheath 1, the thickness 12 may not be uniform around the cross-sectional circumference of the sheath 1. This can be achieved by the outer edge of the sheath 1 having a particular shape. For example, as shown in FIGS. 1a-c, 2a-c, and 3a-c, the outer edge of the sheath 1 in the proximal region 2 and/or distal region 4 may be a polygon, such as a simple polygon. Examples may include, but are not limited to, parallelograms such as a square or rectangle. The polygon may also be a rounded square or rounded rectangle, as shown in FIG. 1b.

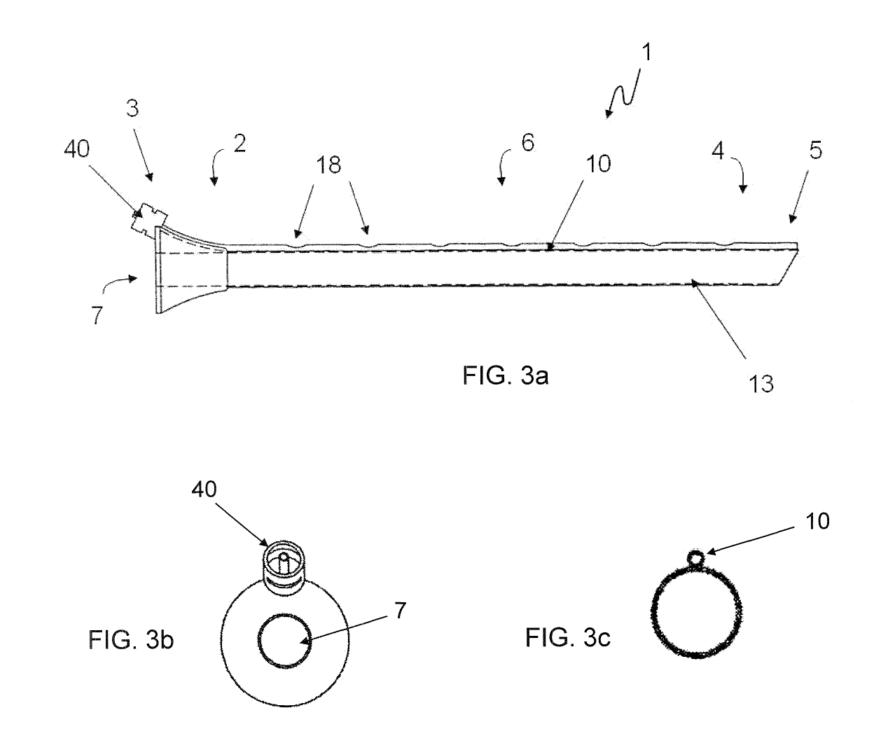

The thickness 12 of the sheath 1 may also differ within a region. For example, as depicted in FIGS. 3a-c, the thickness 12 of the proximal region 2 may gradually increase towards the proximal end 3. This creates the appearance that the proximal end 3 “flares” outward. The channel 10 may thereby be curved in accordance to the curvature of the flare. The curved channel 10 may guide the scope away from the sheath and may be more comfortable for a physician to use. The flare shape may not be too abrupt; otherwise, light fibers in the channel 10 may break.

The proximal end 3 and the distal end 5 of the sheath 1 may be perpendicular to the long axis 14 of the sheath, or may be angled, i.e., beveled. For instance, as shown in FIG. 2a, the proximal end 3 may be perpendicular to the long axis 14, while the distal end 5 may be beveled. The bevel may form an angle 15, for example angle 15 may be between 5 and 85°, or between 30 and 60°.

The distal region 4 may comprise a curvature 16 towards the superior 8 or inferior aspect 9. The curvature 16 may be of a constant radius effecting a 90° turn, but such that the curvature may not cause the light fibers to break.

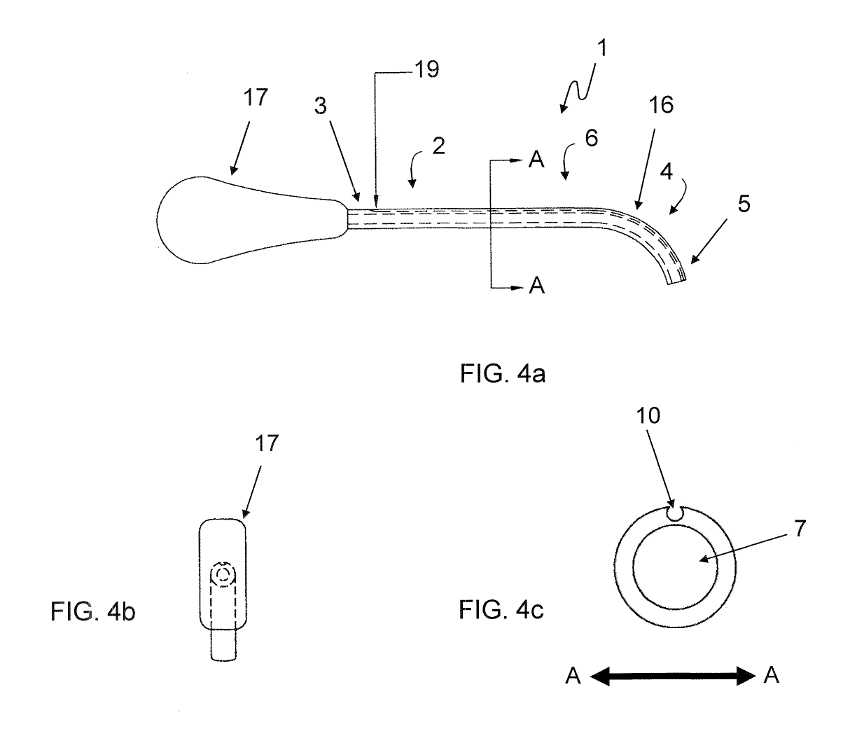

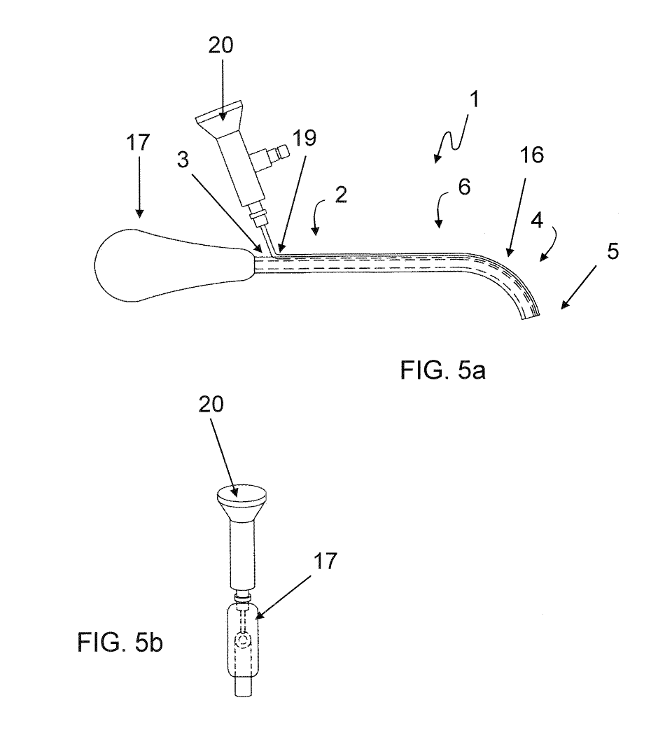

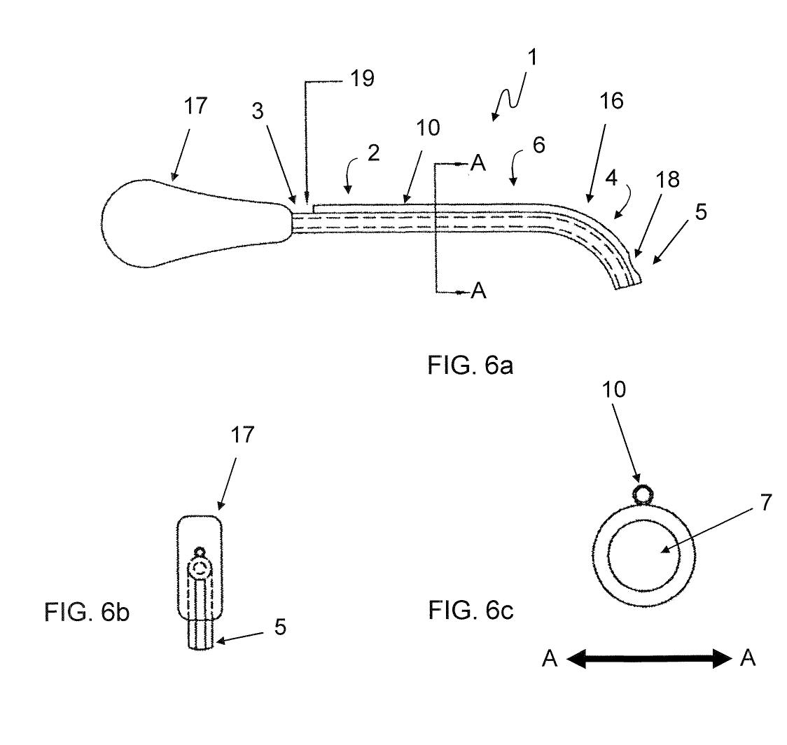

The proximal end 3 may also comprise an end component 17 into which the lumen 7 does not extend, as shown in FIGS. 4a-c and 5a-b. This end component 17 may comprise an ergonomic shape that fits a physician's hand comfortably and may provide ease of handling while the scope is progressed into the airway, such as into the frontal sinus.

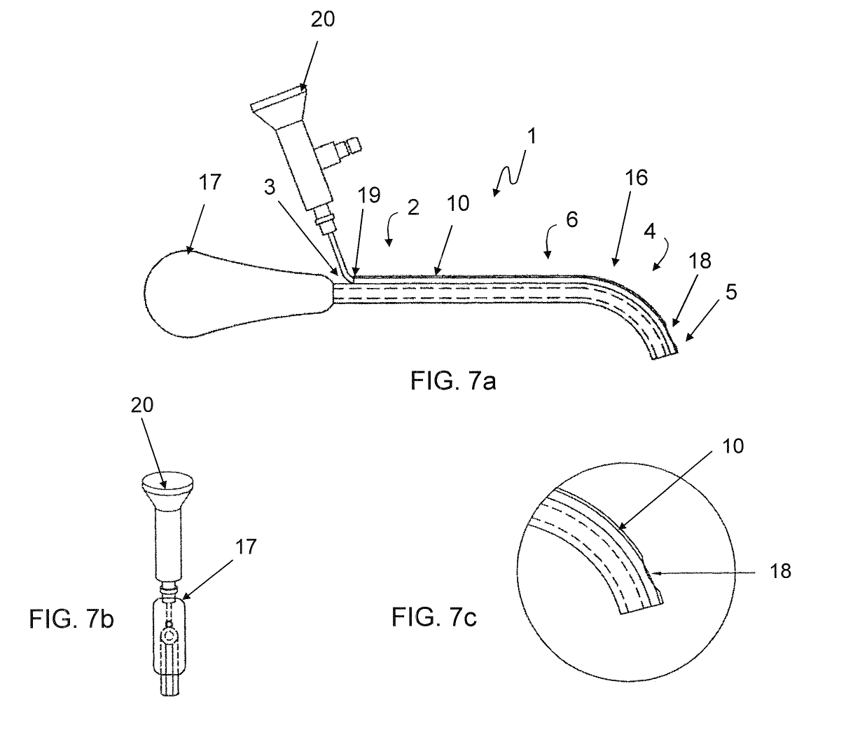

The channel 10 may be inset into the sheath 1 along the superior aspect 8, as shown in FIGS. 1a-c, 2a-c, 4a-c, and 5a-b, or may be a separate enclosed tube attached to the sheath 1 along the superior aspect 8, as shown in FIGS. 3a-c and 7a-c. When the channel 10 is a separate enclosed tube, the channel 10 may have one or more fenestrations 18. The fenestrations 18 may be at a specific location such as in the distal region 4, as shown in FIG. 3a, or the fenestrations 18 may be distributed throughout the length of the sheath, as shown in FIGS. 7a and 7c. The fenestrations can help to provide a sufficient volume of air into the patient's airway, as the fenestrations allow air to flow into the airway.

The delivery device of the present invention may also comprise a fiber scope 20. The outer diameter of the fiber scope 20 may be less than the inner diameter of the channel 10 in order for the fiber scope 20 may fit inside the channel 10. An example of a fiber scope is a bronchoscope.

The fiber scope 20 may be rigid or flexible. The distal end of the fiber scope 20 may comprise a lens. When the fiber scope 20 is in the channel 10, the positioning may be adjusted to ensure that the lens is near or at the distal end 5 of the sheath 1. Notably, the fiber scope may be a zero degree scope.

The fiber scope 20 may enter the channel 10 at a fiber scope insertion site 19, as shown in FIGS. 1a-c, 4a-c, 5a-b, 6a-b, and 7a-c, or may enter the channel 10 at the proximal end 3, as shown in FIGS. 3a-c. The fiber scope 20 may be removable from the channel 10, which may assist in maintaining sterility.

The channel 10 may also be used for ventilation of the patient during use of the device. The ventilation, which may be passive ventilation, may allow a physician to keep, for example, a tracheal stent delivery device in a patient for a long period of time as it stabilizes and controls the patient's breathing. Ventilation also may relieve the physician from having to repeatedly intubate and extubate the patient after the stent is delivered, during confirmation of stent placement, or during repositioning of the stent.

### Tracheal Stent Delivery Device

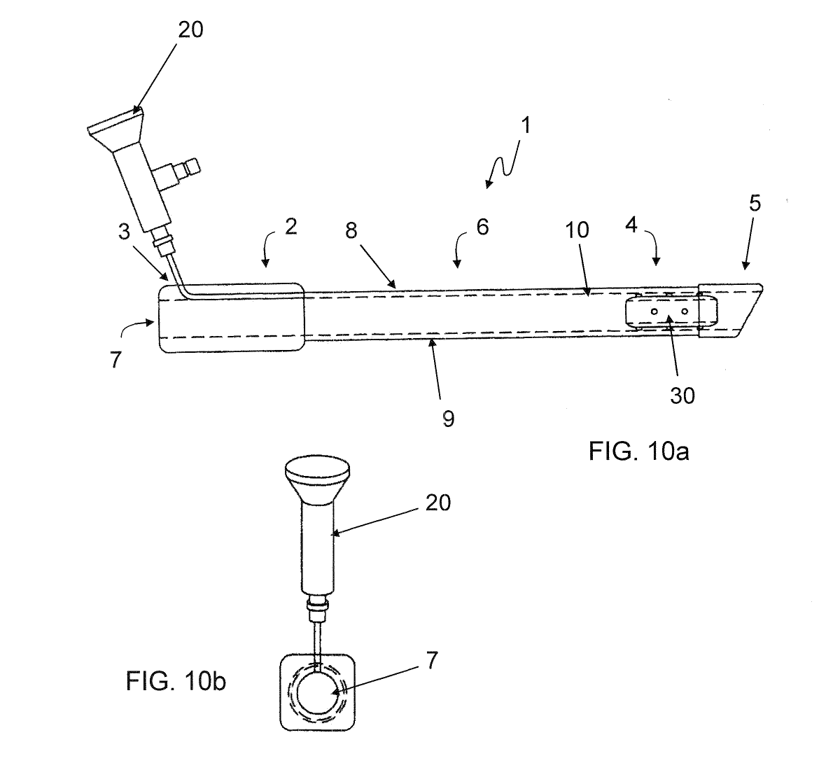

The present invention relates to tracheal stent delivery devices, such as the devices shown in FIGS. 1a-c, 2a-c, 3a-c, 10a-b, 12a-b, and 13a-b. In these embodiments, the lumen may fit the width of a tracheal stent. FIG. 10a-b depicts the sheath 1 of a tracheal stent delivery device having a tracheal stent 30. The tracheal stent 30 may comprise stents as commonly known in the art, or as described in U.S. Pat. No. 7,195,016 and U.S. application Ser. No. 12/559,570, which are incorporated herein by reference.

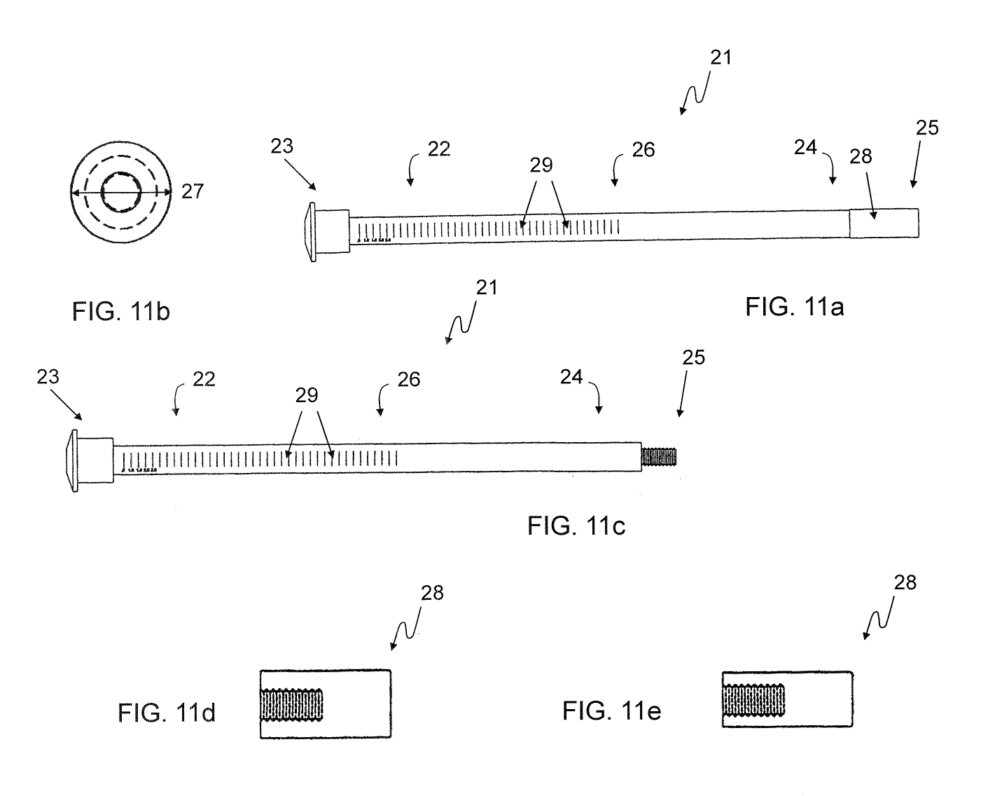

The tracheal stent delivery device may further comprise a cylindrical stent delivery pusher 21, as depicted in FIGS. 11a-e. The stent delivery pusher 21 may comprise a proximal region 22 having a proximal end 23, a distal region 24 having a distal end 25, and a middle region therebetween 26. The stent delivery pusher may be a solid cylinder or a tube. In some embodiments, the stent delivery pusher is a tube, which is lighter and easier to manipulate.

The stent delivery pusher 21 may comprise materials including, but not limited to, nylon, polyvinyl chloride (PVC), or silicone. The length of the stent delivery pusher is approximately the same length as the sheath, i.e., 13-17 inches. The outer diameter 27 of the stent delivery pusher may be less than the diameter of the lumen 7, which allows the stent delivery pusher to fit inside the sheath. Hence, the outer diameter 27 may be about 0.28-0.50 inches. However, the distal end 25 may be wider in diameter so as to provide a surface for pushing the stent delivery pusher. Hence, the distal end 25 will not fit through the lumen 7.

The stent delivery pusher 21 may further comprise removable tips 28, which are attached on the distal end 25. The removable tips 28 may comprise varying shapes and sizes, according to its use. For example, the removable tip 28 may vary in diameter in order to adjust to the diameter of the stent 30. The removable tips 28 may have a diameter greater than the stent delivery pusher 21, but must be less than the diameter of the lumen 7 in order for the removable tip 28 to fit inside the lumen 7. The removable tips 28 may be attached to the stent delivery pusher 21 by a screw (as shown in FIGS. 11c-e), by a snap-fit, by an adhesive, or by other means known in the art.

The stent delivery pusher 21 may also comprise graduated markings 29, such as on the proximal region 22 of the stent delivery pusher 21. The graduated markings 29 may be used as a guide for the user to measure how far the stent delivery pusher 21 is positioned or inserted into the lumen 7. The graduated markings may present standard units, metric units, or both.

### Stent Folding Device

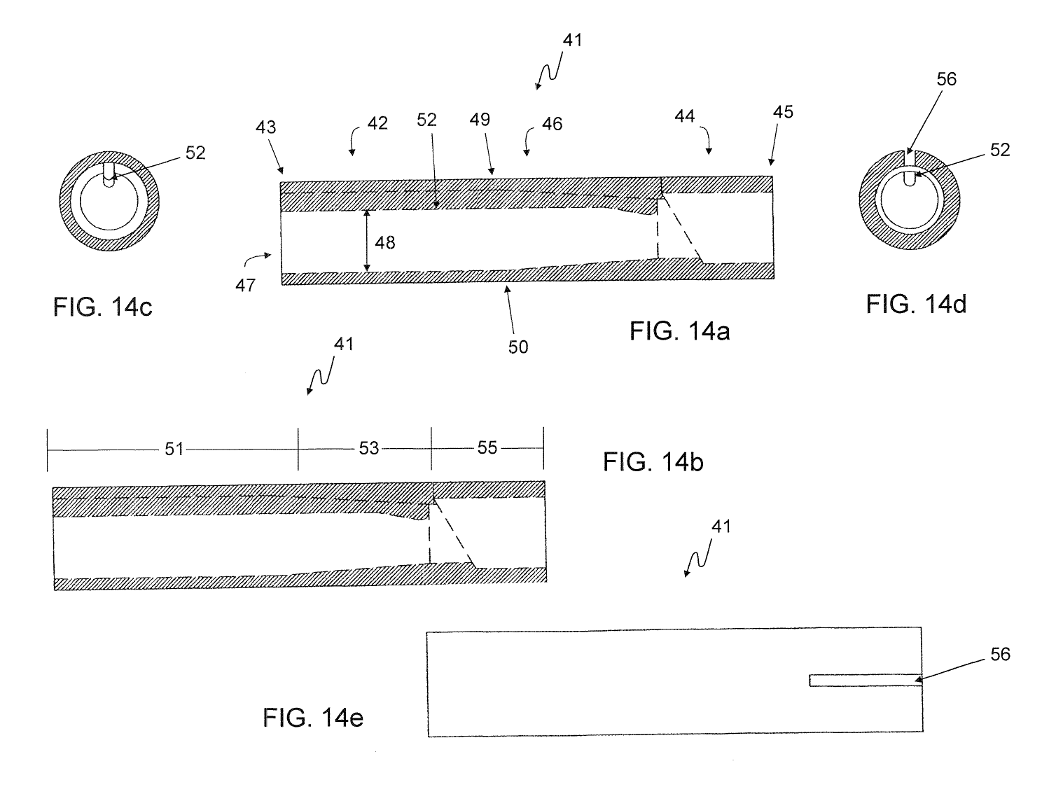

The present invention relates to stent folding devices, as shown in FIGS. 14a-e, 15a-e, and 16a-d, for use with a stent delivery device, such as a tracheal stent delivery device. The stent folding device enables the user to more easily introduce a folded stent into the sheath 1 of a stent delivery device. Current methods often require manually folding the stent and pushing the folded stent into the stent delivery device, which can be difficult.

The stent folding device may comprise a cylindrical sheath 41 used to fold the stent as shown in FIGS. 14a-e and 15a-e. The sheath 41 may comprise a proximal region 42 having a proximal end 43, a distal region 44 having a distal end 45, a middle region therebetween 46; a lumen 47 extending through the sheath 41, a superior aspect 48 of the sheath 41, and an inferior aspect 49 of the sheath 41.

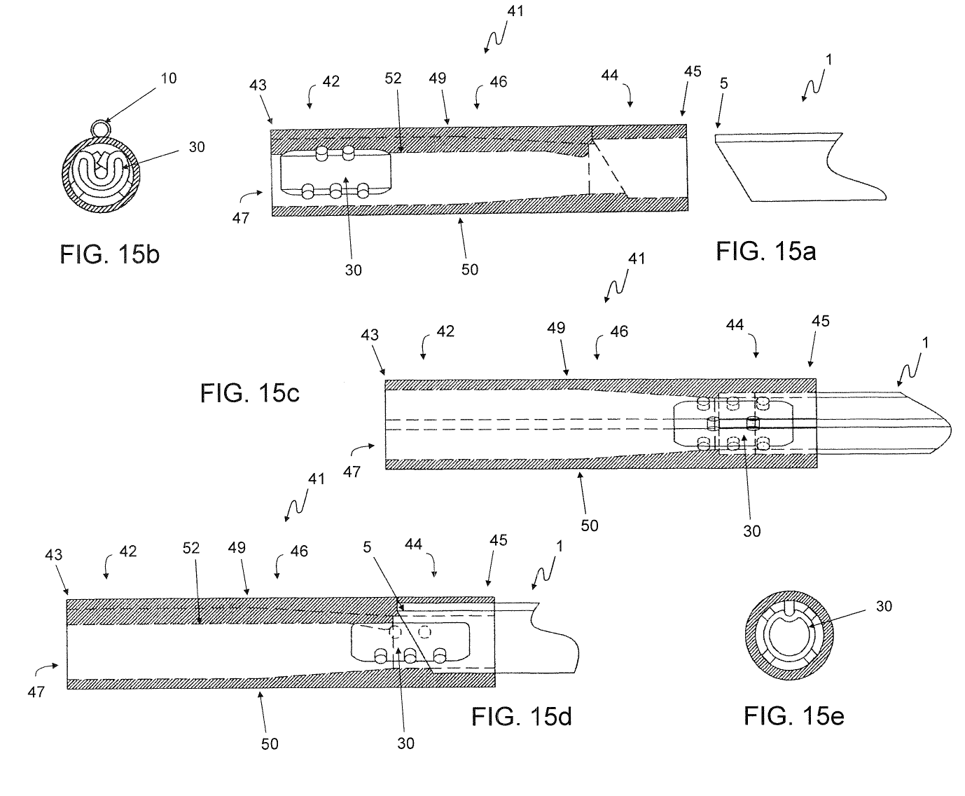

The sheath of the stent folding device can be divided into a first section 51, a second section 53, and a third section 55. The first section 51 holds and positions the stent 30 in the sheath of the stent folding device. One or more guides 52 are positioned in the lumen to ensure that the stent 30 is in the correct orientation before it progresses to the second section 53. The one or more guides may extend from the wall of the lumen. In certain embodiments, the one or more guides may be shaped like fin(s). In some embodiments wherein the stent 30 may comprises posts extending radially from its outer surface as shown in FIGS. 15a-e, the guide(s) 52 will interact with the posts to orient the stent 30. The posts, for instance, may rest on either side of the fin.

The lumen 47 in the second section 53 comprises a smaller diameter 48 than the first section. The fin(s) expands downward, which forces the stent 30 to fold more tightly into a diameter 48 that is similar to the diameter 11 of the lumen 7 of the sheath 1 of the stent delivery device.

The third section 55 comprises the lumen 47 having an internal shape and diameter 48 that is configured to fit the external shape of the distal end 5 of the sheath 1 of the stent delivery device. A groove 56 in the wall of the third section 55 mirrors the diameter of the distal end of the scope channel tube 10 of the stent delivery device. The groove 56 can thereby fit the scope channel tube 10 as the distal end 5 of the sheath 1 of the stent delivery device is fit within the third section 55 of the sheath 41 of the stent folding device, and can align the distal end 5 of the sheath 1 of the stent delivery device with the internal shape of the third section 55 of the sheath 41 of the stent folding device.

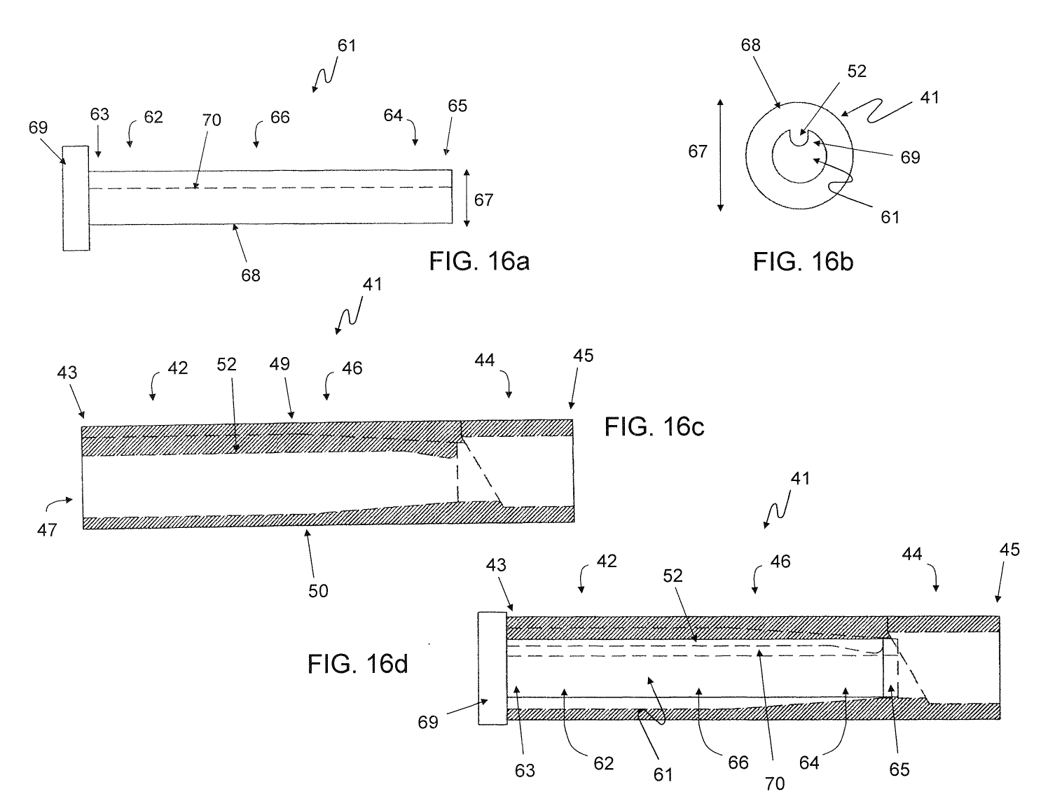

The stent folding device may also comprise a stent folding pusher as shown in FIGS. 16a-d. The stent folding pusher may comprise cylinder 61 which may comprise a proximal region 62 having a proximal end 63, a distal region 64 having a distal end 65, a middle region therebetween 66, an outer diameter 67, and an outer surface 68. In some embodiments, the stent folding pusher may be a tube with a lumen rather than a cylinder.

The stent folding pusher may comprise a disc-shaped handle 69 on the proximal end 63, which allows the user to effective apply the appropriate force necessary to push the stent in. The stent folding pusher may also comprise a groove 70 on the outer surface 68 that may be shaped to fit the guide 52 in the tube 41. The interaction of the groove 70 of the stent folding pusher with the guide 52 may direct the stent folding pusher steadily as it is forced up against the stent 30 and continues to move the stent forward.

### Stent Loading Device

The present invention relates to stent loading devices for use with a stent folding device and a stent delivery device, such as a tracheal stent delivery device. The stent loading device enables the user to move the folded stent from the sheath of a stent folding device to the sheath of a stent delivery device. The stent loading device may be used to insert the stent into either the proximal end or the distal end of the sheath of the stent delivery device. In certain embodiments, the stent loading device may be used to insert the stent into the proximal end of the sheath of the stent delivery device, which may be advantageous. Before inserting a stent into the airway of a patient, a physician may insert the sheath of the stent delivery device to evaluate the patient's airway. If a stent can only be loaded into the stent delivery device at the distal end of the sheath, the sheath would have to be withdrawn after the evaluation to load the stent and then be re-inserted into the airway of the patient; this repeated insertion and withdrawal of the sheath of the stent delivery device can cause trauma to the airway. Therefore, by loading the stent into the sheath of the stent delivery device through its proximal end, the stent delivery device does not have to be withdrawn following the evaluation of the airway.

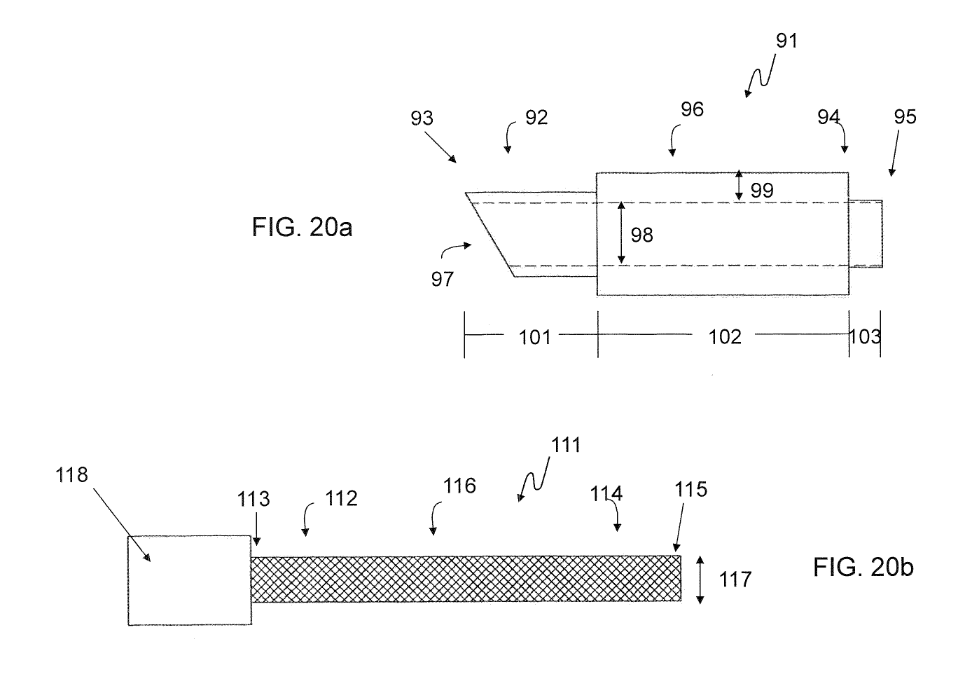

As shown in FIG. 20a, the stent loading device may comprise a circular sheath 91 that may comprise a proximal region 92 having a proximal end 93, a distal region 94 having a distal end 95, a middle region therebetween 96, and a lumen 97 extending through the sheath 91. The lumen 97 may comprise a diameter 98 appropriate for the diameter of folded stents. The length of the sheath 91 can be determined by one of ordinary skill in the art. The sheath may also comprise a thickness 99, i.e., distance from the outer edge of the lumen 97 to the outside of the sheath, as shown in FIG. 20a.

The sheath 91 of the stent loading device may be divided into a first section 101, a second section 102, and a third section 103. The first section 101 may comprise a shape configured to fit within the lumen 47 of third section 55 of the sheath 41 of the stent folding device. The second section 102 of the sheath 91 of the stent loading device may comprise a greater thickness than the first section 101 and the third section 103, and may be used to handle the sheath 91 by the user. The third section 103 of the sheath 91 may comprise a shape configured to fit within the lumen 7 at either the proximal end 3 or the distal end 5 of the sheath 1 of the stent delivery device. In certain embodiments, the third section 103 may comprise a shape configured to fit within the lumen 7 at the proximal end 3 of the sheath 1 of the delivery device.

The stent loading device may also comprise a cylindrical stent loading pusher 111. A stent loading pusher 111, as shown in FIG. 20a, may be used with the stent sheath 91 of the stent loading device to move a stent through the lumen 97 of the sheath 91. The stent loading pusher 111 may comprise a proximal region 112 having a proximal end 113, a distal region 114 having a distal end 115, and a middle region therebetween 116. In some embodiments, the stent folding pusher may be a tube with a lumen rather than a cylinder. The stent loading pusher may also comprise a handle 118 on the proximal end 113, which allows the user to effective apply the appropriate force necessary to push the stent in.

The outer diameter 117 of the stent loading pusher may be less than the diameter of the lumen 97 of the sheath of the stent loading device, which allows the stent loading pusher to fit inside the lumen 97. However, the distal end 115 may be wider in diameter so as to provide a surface for pushing the stent delivery pusher. Hence, the distal end 115 will not fit through the lumen 97.

### Sinus Catheter Delivery Device

The present invention relates to sinus catheter delivery devices, as shown in FIGS. 4a-c, 5a-b, 6a-b, and 7a-c. The distal region 4 of the sheath 1 may comprise a curvature, wherein the degree of curvature is appropriate for the sinus cavity.

As described above, the proximal end 3 may also comprise an end component 17, which comprises an ergonomic shape that fits a physician's hand comfortably and provides ease of handling while the scope is progressed up to and into the frontal sinus. The shape may allow for thumb and forefinger manipulation.

In some embodiments, fenestrations 18 may be located at the distal end of the channel 10. The fenestrations 18 may help to suction the sinus as a means of lavage, and may help position a fiber scope into the main lumen of the device.

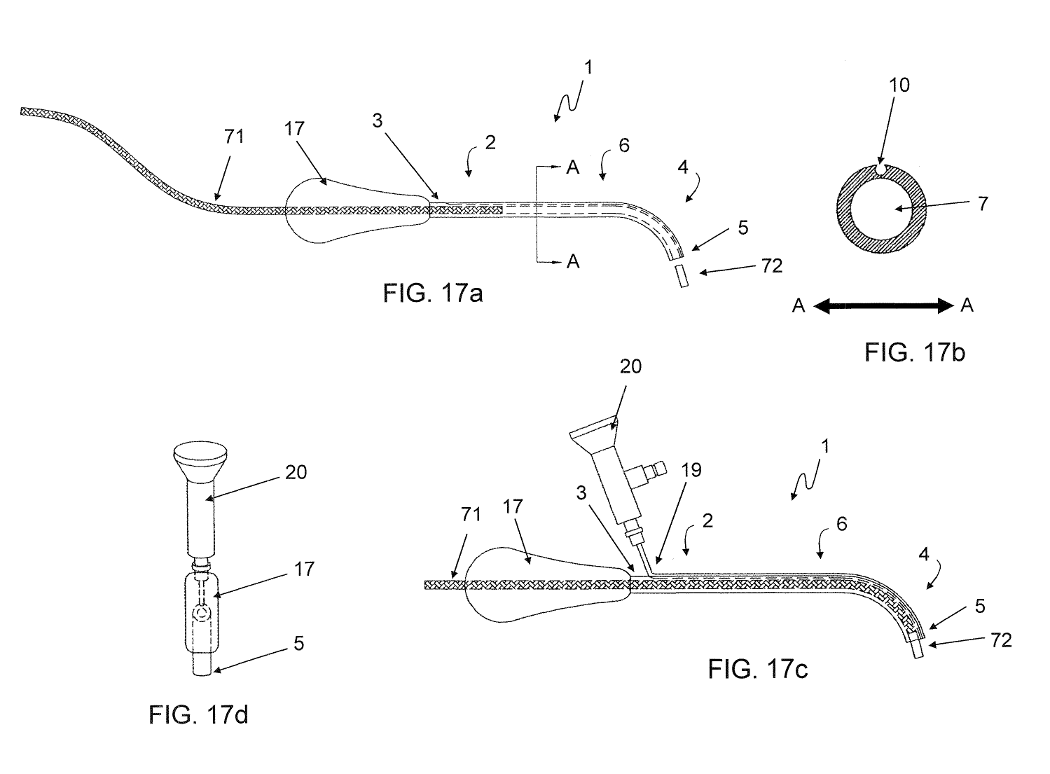

In other embodiments, the sinus catheter delivery device may further comprise a sinus catheter pusher 71 as shown in FIGS. 17a-c. The sinus catheter pusher 71 helps slide the catheter 72 through the sinus catheter delivery device to position the catheter 72 within the sinus. The scope 20 can be used with the sinus catheter pusher 71.

### ALTERNATIVE EMBODIMENTS

In certain embodiments, the fiber scope 20 may be removed from the channel 10 and a ventilator (not shown) may be attached to the proximal end 40 of the channel 10. This allows for ventilation of the patient and limits the number of time that the patient is extubated and intubated.

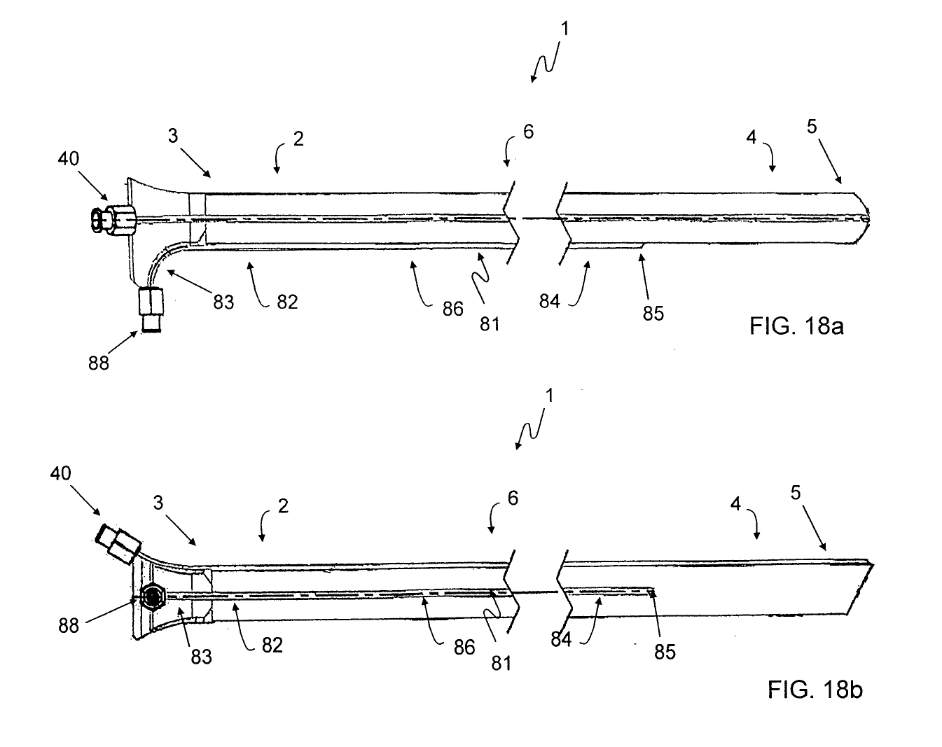

In other embodiments, a separate ventilation tube 81 may extend along the outside of the delivery device, as shown in FIGS. 18a-b. The ventilation tube may comprise a proximal region 82 having a proximal end 83, a distal region 84 having a distal end 85, a middle region therebetween 86, and a lumen 87 extending through the ventilation tube 81. The distal end 85 ventilation tube 81 may extend to the distal end 5 of the delivery device, or may only extend part-way. The proximal end 83 of the ventilation tube 81 may comprise a connection 88 to a ventilator circuit (not shown) to connect to a ventilation device as known in the art.

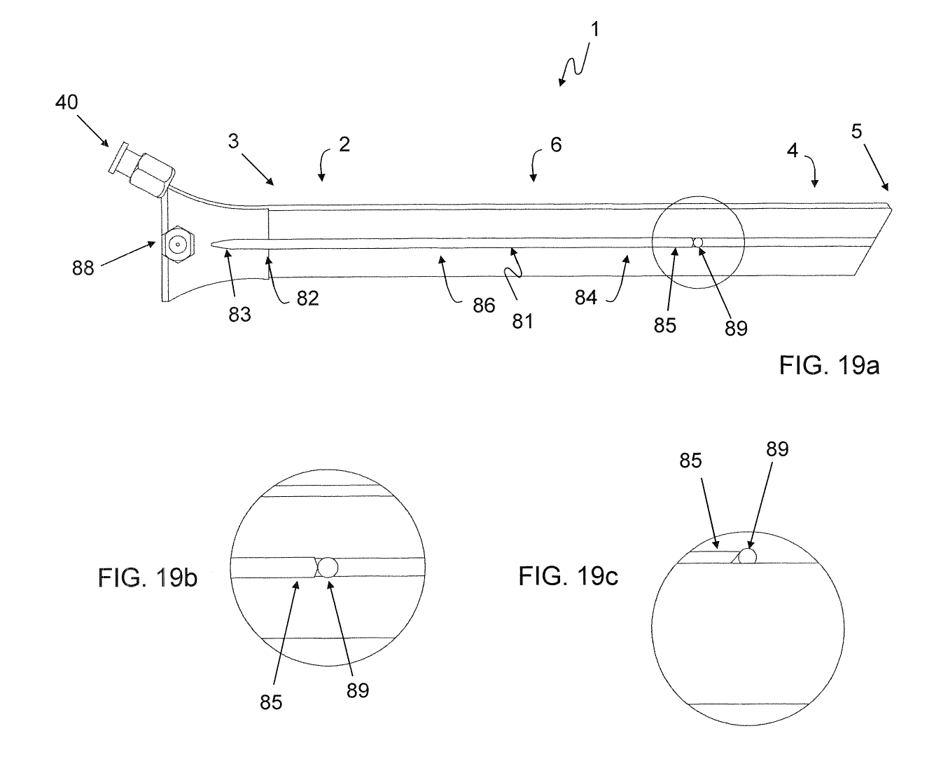

In certain embodiments, as shown in FIGS. 19a-c, the distal end 85 of the ventilation tube 81 may be blunted. For example, this feature may reduce the possibility of damage to the airway during the introduction of the tracheal introducer tube. The blunt end may be formed by an attachment 89 to the end of the ventilation tube. This attachment, as an example, may be a stainless steel bead welded onto the distal end 85.

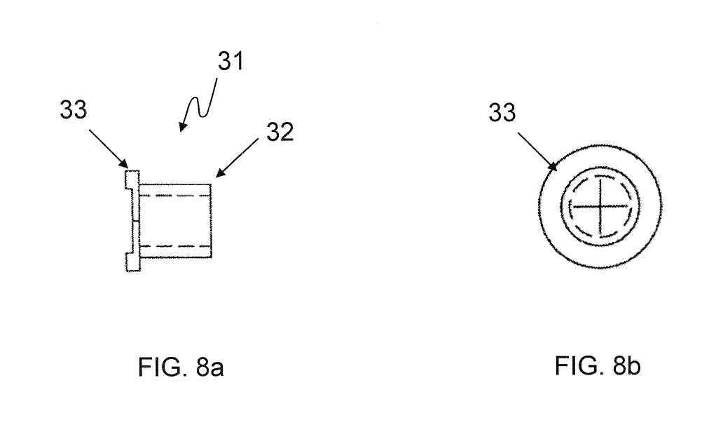

The present invention may further comprise a rubber cap 31, which fits on the proximal end 3 of the sheath 1, as shown in FIGS. 8a-b and 9a-b. The rubber cap 31 may comprise a cylindrical insert 32 and a disc top 33. The rubber cap 31 fits onto the proximal end 3 of the sheath 1 by placing the cylindrical insert 32 into the lumen 7.

The rubber cap may also comprise one or more slits 34, which create an air seal while allowing a bronchoscope to pass through as it is introduced into the lumen of the device. The one or more slits may be placed around the circumference of the cap. For instance, there may be two slits placed 90° apart, relative to each other.

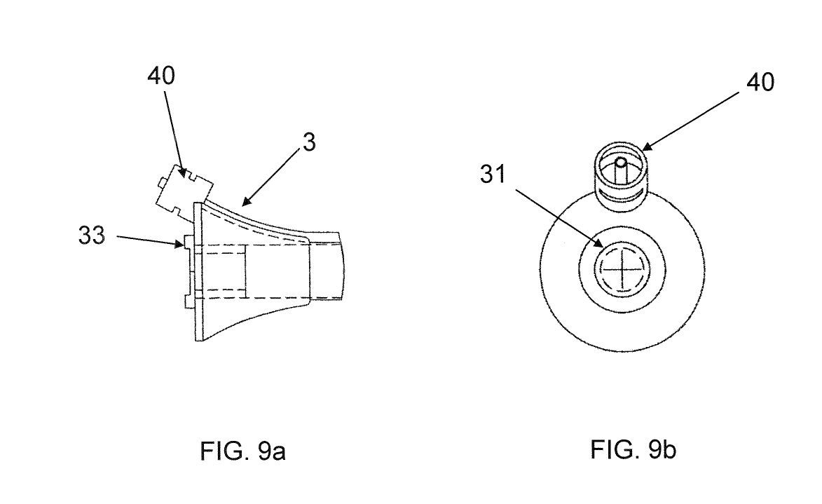

The rubber cap 31 can be used in instances in which the device is maintained in the patient for an extended period of time, such as when a ventilator is attached to the device. The device may be in the patient for a minimal period of time in order to prevent any obstruction to the patient's ability to breathe. In order to appropriately ventilate the patient, the rubber cap 31 can be placed onto the proximal end 3 of the sheath 1 of the device as shown in FIG. 9b. The rubber cap 31 can act as a means of preventing the ventilated air from passing through the device, rather than into the lungs. Once the patient is ventilated, such as through the channel 10, the stent can be viewed in position by passing a bronchoscope through the center of the device while ventilating the patient through the channel 10.

### Methods of Using the Devices

The present invention relates to methods of using the devices described herein. The stent delivery devices of the present invention may be used to deliver a stent to an airway in a patient. For example, in some embodiments, the stent delivery device may be used to place a tracheal stent at a particular site, such as a stenosis, in a patient by a method comprising (1) placing the device according to embodiments of the present invention in the airway of the patient, wherein the device contains the stent; (2) positioning the distal end of the device at site in the airway by: (a) viewing the inside of the airway with the bronchoscope; and (b) moving the instrument in the airway until the distal end is at the site; (3) depositing the stent at the site in the airway; and (4) removing the instrument from the airway. The method may further comprise a step of repositioning the stent if it is not in the appropriate position, before the instrument is removed from the airway.

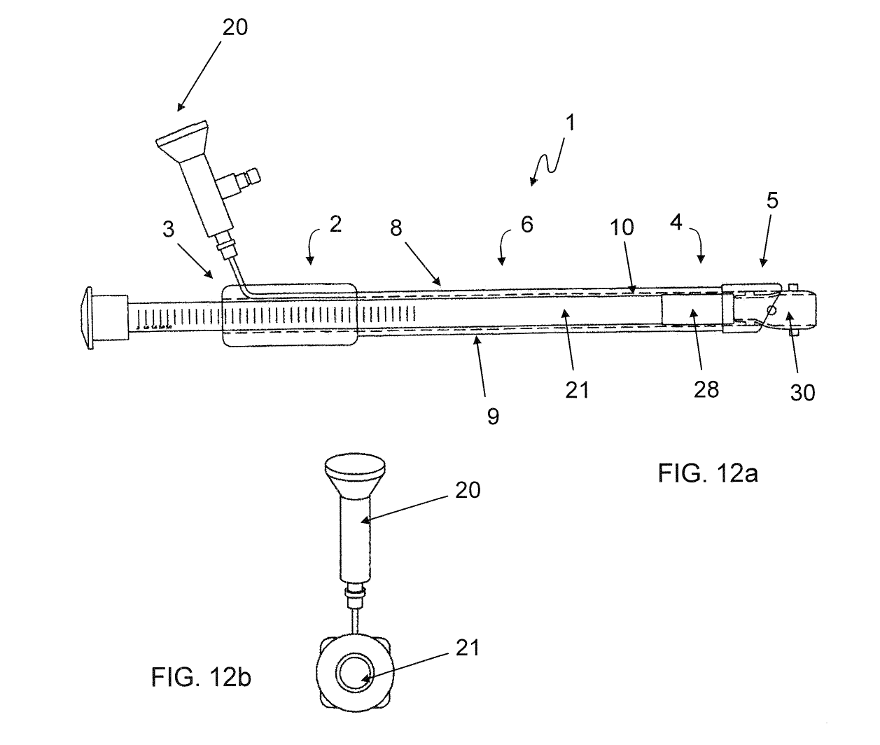

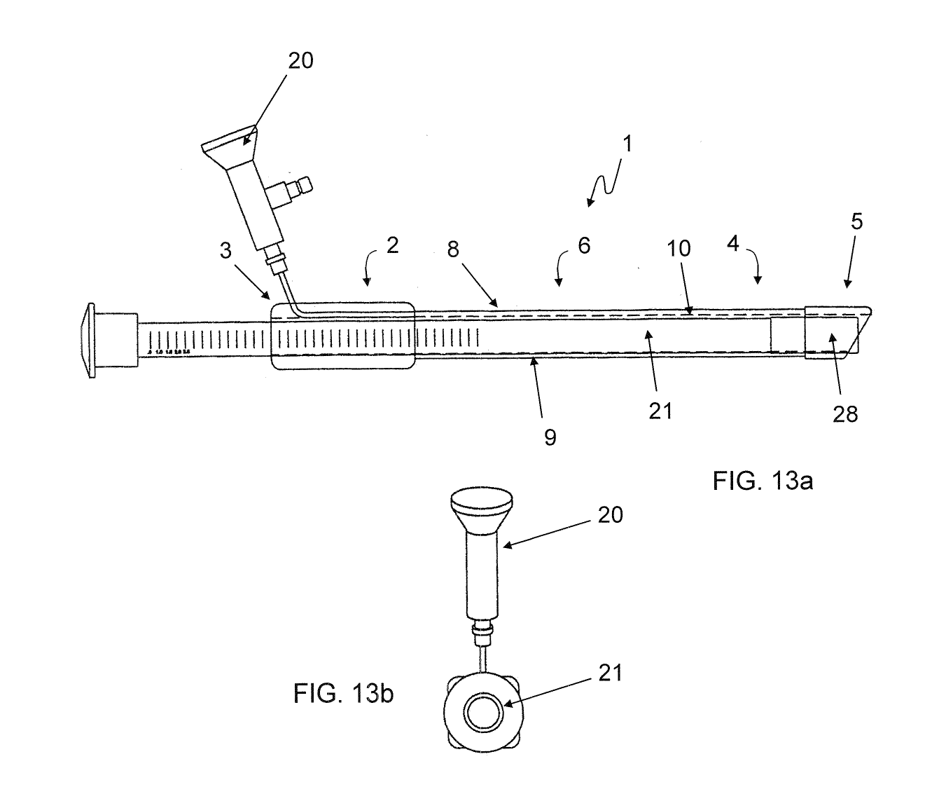

The stent may be deposited using the stent delivery device. FIGS. 10a-b show a stent delivery device having a stent 30 in the distal region 4 of the sheath 1. A stent delivery pusher 21 may be inserted into the lumen 7 of the sheath 1 from the proximal end 3, as shown in FIGS. 12a-b. Pressure is applied to the stent delivery pusher 21 to push the stent 30 through the distal end 5 of the device as shown in FIGS. 13a-b, thereby depositing the stent 30 outside of the stent delivery device.

In embodiments wherein the stent is being used to treat a stenosis, prior to insertion of the stent into the airway, the stenosis may be dilated with conventional flexible dilators. In addition, a water soluble gel, such as K-Y JELLY® or the like, may be applied to the stent prior to its placement in the device. Alternatively, the stent may be coated with a silicone spray. Once the stent is delivered to the stenosis, the user can confirm its placement with the fiber scope, repositioning the stent if necessary, and the device can be removed.

Stent folding devices of the present invention may be used to insert a stent into stent delivery devices. Embodiments of a method of folding the stent and inserting the folded stent into the sheath of the stent delivery device is shown in FIGS. 15a-e. The method may comprise positioning an end of the sheath 1 of the stent delivery device within the sheath 41 of the stent folding device (in this case, within the third section 55 of the sheath 41). The stent 30 may be inserted into the free end of the sheath 41 (in this case, proximal end 43, which is in first section 51), as shown in FIGS. 15a-b. A stent folding pusher may then be inserted into the free end of the sheath 41 and pressure may be applied to the stent folding pusher to move the stent 30 through the sheath 41 and cause it to fold because of the guide 52 that changes the stent's shape (in this case, the guide is in the second section 53 of the sheath 41). Pressure may continue to be applied to push the stent 30 into the sheath 1 of the stent delivery device (FIGS. 15c-e).

In some embodiments, the distal end 5 of the sheath 1 of the stent delivery device may be positioned within the sheath 41 of the stent folding device. In certain embodiments, the end of the sheath 1 of the stent delivery device may be positioned within the distal end 45 of the sheath 41 of the stent folding device, such that the proximal end 43 of the sheath 41 of the stent folding device is the free end.

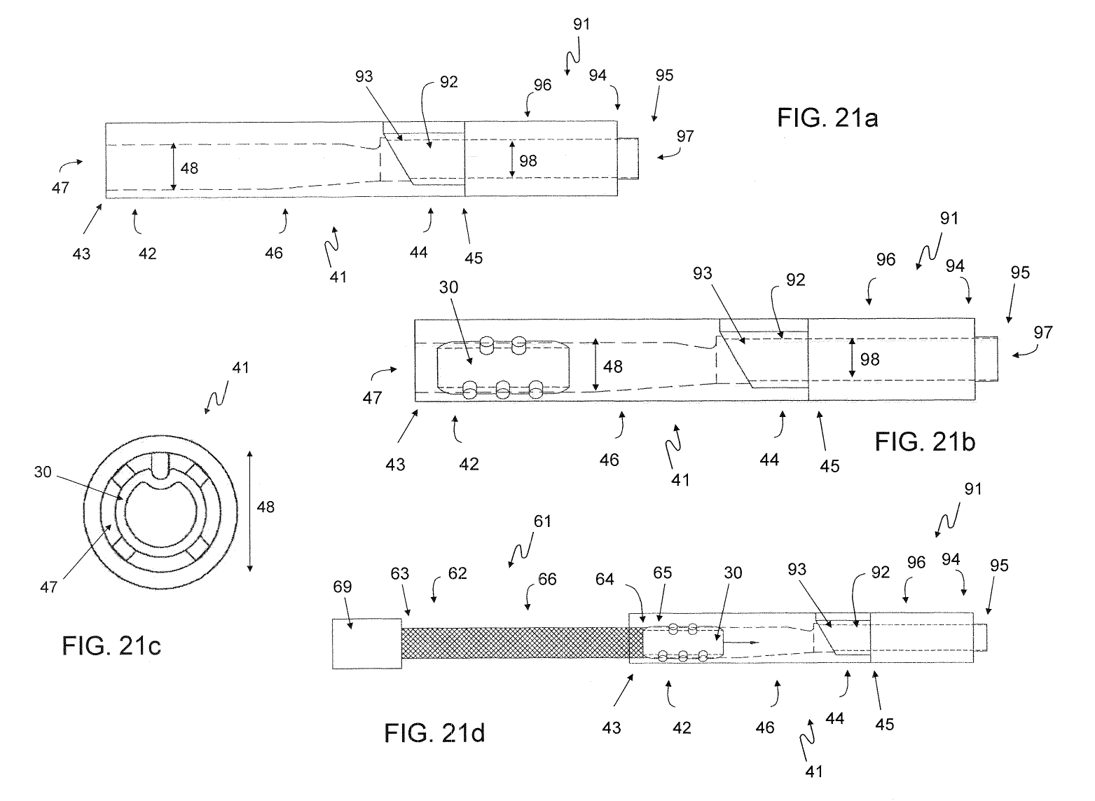

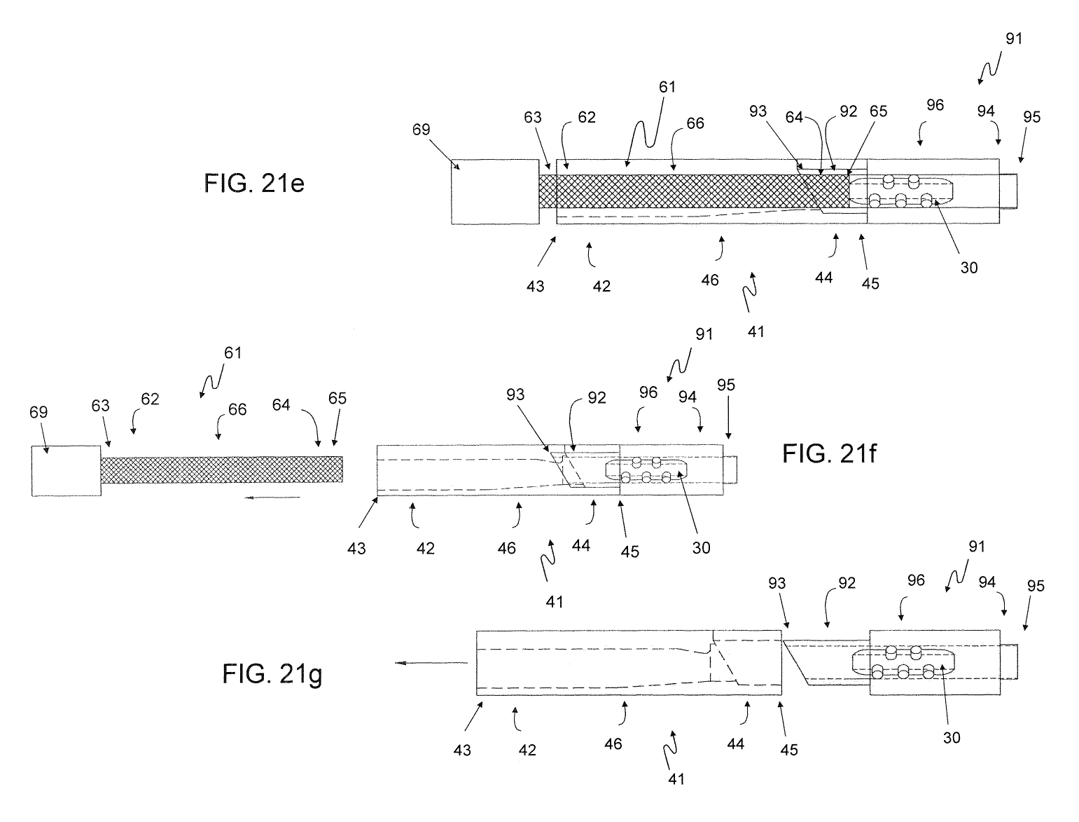

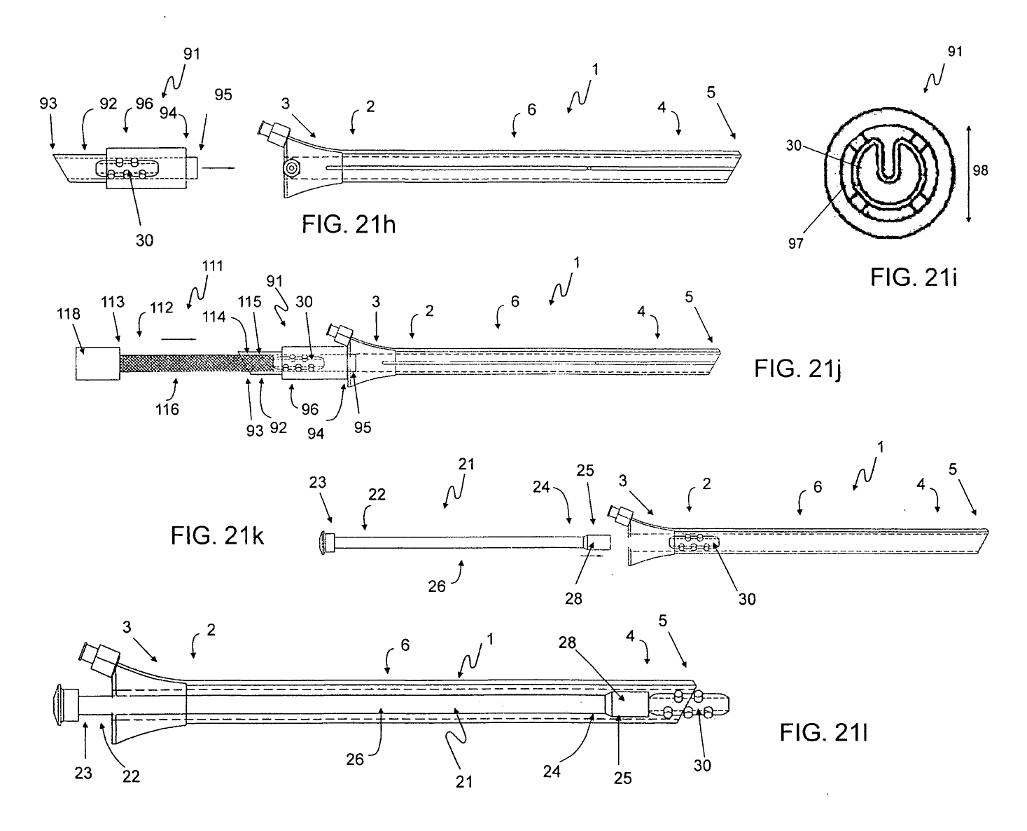

Stent loading devices of the present invention may be used to insert a stent into stent delivery devices. Embodiments of a method of using a stent loading device to move a folded stent from the stent folder to the stent delivery device is shown in FIGS. 21a-l. The method may comprise positioning an end (in this case, the proximal end 93, which is the first section 101) of the sheath 91 of the stent loading device within the sheath 41 of the stent folding device (FIG. 21a). The stent 30 may be inserted into the free end of the sheath 41 of the stent folding device (FIGS. 21b-c). A stent folding pusher 21 may then be inserted into the free end of the sheath 41 (in this case, the proximal end 43), as shown in FIG. 21d, and pressure may be applied to the stent folding pusher 21 to move the stent 30 through the sheath 41 of the stent folding device, causing it to fold because of the guide 52 that changes the stent's shape, and to progress the stent 30 into the sheath 91 of the stent loading device (FIG. 21e). The sheath 91 of the stent loading device may then be separated from the sheath 41 of the stent folding device (FIG. 21g), and the sheath 91 of the stent loading may be positioned so that an end (in this case, the distal end 95, which is the third section 103) of the sheath 91 of the stent loading device is within an end (n this case, proximal end 3) of the sheath 1 of the stent delivery device (FIGS. 21h-i). A stent loader pusher 111 may be inserted into the free end (n this case, the proximal end 93) of the sheath 91 of the stent loading device (FIG. 21j) and pressure may be applied to the stent loader pusher 111 to move the stent 30 through the sheath 91 (and through the second section 102) of the stent loading device and into the sheath 1 of the stent delivery device. The sheath 91 of the stent loading may then be separated from the sheath 1 of the stent delivery device (FIG. 21k),

In some embodiments, the proximal end 93 of the sheath 91 of the stent loading device is positioned within the sheath 41 of the stent folding device. In certain embodiments, the end of the sheath 91 of the stent loading device is positioned within the distal end 45 of the sheath 41 of the stent folding device, such that the proximal end 43 of the sheath 41 of the stent folding device is the free end. In some embodiments, the distal end 95 of the sheath 91 of the stent loading device is positioned within the sheath 1 of the stent delivery device, such that the proximal end 93 of the sheath 91 of the stent loading device is the free end. In certain embodiments, the end of the sheath 91 of the stent loading device is positioned within the proximal end 3 of the sheath 1 of the stent delivery device.

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention. The full scope of the invention should be determined by reference to the claims, along with their full scope of equivalents, and the specification, along with such variations.

What is claimed is:

1. A device for delivering a stent to a site in the airway of a patient in need thereof, comprising:
(a) a sheath comprising:
(i) a proximal region;
(ii) a distal region;
(iii) a middle region therebetween,
(iv) a lumen extending through the sheath, wherein the lumen comprises a diameter;
(v) a superior aspect comprising a channel extending the length of the sheath, wherein the channel comprises a diameter; and
(vii) an inferior aspect; and
(b) an instrument comprising an outer diameter, wherein the outer diameter of the instrument is less than the diameter of the channel to allow the instrument to fit inside the channel.

2. The device of claim 1, wherein the instrument is selected from the group consisting of a fiber scope and a ventilator.

3. The instrument of claim 2, wherein the fiber scope is a bronchoscope.

4. The instrument of claim 2, wherein the fiber scope is rigid or flexible.

5. The device of claim 1, wherein the diameter of the lumen of the sheath can fit a tracheal stent.

6. The device of claim 1, wherein the distal region comprises a beveled distal end.

7. The device of claim 1, wherein the sheath comprises stainless steel.

8. The device of claim 1, further comprising a stent delivery pusher comprising (i) a proximal region; (ii) a distal region; and (iii) a middle region therebetween, and (iv) an outer diameter, wherein the outer diameter of the stent delivery pusher is less than the diameter of the lumen of the sheath to allow the stent delivery pusher to fit inside the lumen of the sheath.

9. The device of claim 8, wherein the stent delivery pusher is a rod or tube.

10. The device of claim 8, wherein the stent delivery pusher comprises removable tips.

11. The device of claim 10, wherein the tips are attached to the stent delivery pusher by screw, snap-fit, or adhesive.

12. The device of claim 10, wherein the tips comprise an outer diameter greater than the outer diameter of the stent delivery pusher, and the outer diameter is less than the diameter of the lumen of the sheath.

13. The device of claim 8, wherein the stent delivery pusher comprises graduated markings.

14. The device of claim 1, wherein the inner diameter of the lumen of the sheath can accommodate the outer diameter of a sinus stent

15. The instrument of claim 14, wherein the sheath comprises a curvature.

16. The instrument of claim 14, wherein the curvature is in the distal region.

17. The instrument of claim 14, wherein the channel is within the superior aspect of the sheath.

18. The instrument of claim 14, wherein the channel is outside of the superior aspect of the sheath.

19. A method of delivering a stent to a site in an airway of a patient in need thereof, comprising:
(1) placing the device of claim 1 in the airway of the patient, wherein a stent is in the lumen of the sheath of the device;
(2) positioning the distal end of the instrument of the device inside the airway by:
(a) viewing the inside of the airway with the fiber scope; and
(b) moving the instrument in the airway until the distal end of the instrument is adjacent to the site in the airway at which the stent is to be placed;
(3) depositing the stent at the site in the airway; and
(4) removing the instrument from the airway.

20. The method of claim 19, wherein the stent is deposited by a stent delivery pusher inserted into the sheath of the stent delivery device.

21. The method of claim 20, wherein the stent delivery pusher moves the stent out of the sheath of the stent delivery device

22. The method of claim 20, wherein graduated markings on the stent delivery pusher guide how far to insert the stent delivery pusher into the sheath of the stent delivery device.

23. The method of claim 19, wherein the site in the airway is a stenosis.

24. A stent folding device for folding a stent for placement into a stent delivery device, comprising:
(a) a sheath comprising:
(i) a proximal region;
(ii) a distal region;
(iii) a middle region therebetween,
(iv) a lumen extending through the sheath, wherein the lumen comprises a diameter; wherein the lumen in the distal region of the sheath is configured to receive an end of a stent delivery device; and wherein the diameter of the lumen in the proximal region of the sheath is greater that the diameter of the lumen in the middle region of the sheath.
(v) one or more fins inside the lumen; and
(b) a cylindrical stent folding pusher comprising
(i) an outer diameter that is less than the diameter of the lumen;
(ii) an outer surface;
(iii) and a groove on the surface that is configured to fit the one or more fins.

25. A method of inserting a stent into a stent delivery device using the stent folding device of claim 24, comprising:
(1) inserting an end of the stent delivery device into the lumen of the sheath of the stent folding device, wherein the end of the stent delivery device is inserted into the distal region of the sheath;
(2) inserting the stent into the lumen of the sheath, wherein the stent is inserted into the proximal region of the sheath;
(3) moving the stent through the lumen from the proximal region of the sheath to the distal region of the sheath and through the end of the stent delivery device;
(4) removing the end of the stent delivery device from the lumen of the sheath of the stent folding device.

26. A stent loading device for loading a folded stent into a stent delivery device, comprising:
(a) a sheath comprising:
(i) a proximal region;
(ii) a distal region;
(iii) a middle region therebetween,
(iv) a lumen extending through the sheath, wherein the lumen comprises a diameter; wherein the proximal region of the sheath is configured to be inserted into a stent folding device; wherein the lumen in the distal region of the sheath is configured to receive an end of a stent delivery device; and wherein the diameter of the lumen in the proximal region of the sheath is greater that the diameter of the lumen in the middle region of the sheath.
(b) a cylindrical stent loading pusher comprising an outer diameter that is less than the diameter of the lumen.

27. A method of transporting a folded stent from a stent folding device to a stent delivery device using the stent loading device of claim 26, comprising:
(1) inserting the proximal region of the sheath of the stent loading device into a stent folding device, wherein the stent folding device contains a folded stent;
(2) moving the folded stent from the stent folding device into the proximal region of the sheath;
(3) inserting an end of the stent delivery device into the lumen of the sheath, wherein the end of the stent delivery device is inserted into the distal region of the sheath;
(4) pushing the stent through the lumen of the sheath from the proximal region to the distal region of the sheath and through the end of the stent delivery device;
(5) removing the end of the stent delivery device from the lumen of the sheath of the stent loading device.
